(19) United States
(12) Patent Application Publication
(10) Pub. No.: US 2022/0323435 A1
(43) Pub. Date: Oct. 13, 2022

(54) COMPOSITION AND METHOD FOR TREATMENT OF DEPRESSION AND PSYCHOSIS IN HUMANS

(71) Applicant: Glytech LLC, Ft. Lee, NJ (US)

(72) Inventor: Daniel C. Javitt, Ft. Lee, NJ (US)

(73) Assignee: Glytech LLC, Ft. Lee, NJ (US)

(21) Appl. No.: 17/844,087

(22) Filed: Jun. 20, 2022

Related U.S. Application Data
(63) Continuation of application No. 16/166,101, filed on Oct. 21, 2018, now abandoned.
Application No. 16/166,101 is a continuation-in-part of application No. 15/650,912, filed on Jul. 16, 2017, now Pat. No. 10,660,887.
Application No. 15/650,912 is a continuation of application No. 13/936,198, filed on Jul. 7, 2013, now Pat. No. 9,737,531.
Application No. 16/166,101 is a continuation-in-part of application No. 15/987,932, filed on May 24, 2018, now Pat. No. 10,583,138.
Application No. 16/166,101 is a continuation-in-part of application No. 15/723,391, filed on Oct. 3, 2017, now abandoned.
Application No. 15/723,391 is a continuation of application No. 13/982,460, filed on Sep. 29, 2013, now Pat. No. 9,789,093.
(60) Provisional application No. 61/741,114, filed on Jul. 12, 2012.
(60) Provisional application No. 61/741,115, filed on Jul. 12, 2012.
(60) Provisional application No. 62/518,020, filed on Jun. 12, 2017.
(60) Provisional application No. 61/437,700, filed on Jan. 31, 2011.
(60) Provisional application No. 61/494,907, filed on Jun. 9, 2011.

(51) Int. Cl. A61K 31/496; A61K 31/4525; A61K 31/135; A61K 31/431; A61K 45/06; A61K 31/138; A61K 31/55; A61K 31/554; A61K 31/06; A61K 31/519; A61K 31/553; A61K 31/381; A61K 31/42; A61K 31/551; A61K 9/00; A61K 31/165; A61K 31/495; A61K 31/137; A61K 31/343

(52) U.S. Cl. CPC A61K 31/496; A61K 31/4525; A61K 31/135; A61K 31/431; A61K 45/06; A61K 31/138; A61K 31/55; A61K 31/554; A61K 31/06; A61K 31/519; A61K 31/553; A61K 31/381; A61K 31/42; A61K 31/551; A61K 9/00; A61K 31/165; A61K 31/495; A61K 31/137; A61K 31/343; A61K 2300/00; A61K 9/08

(57) ABSTRACT

This application relates to combination compositions for use in treatment of depression, comprising an effective amount of an N-methyl-D-aspartate receptor (NMDAR) antagonist combined with an anti-depressant agent or an atypical antipsychotic approved for treatment of depression. In a preferred embodiment, D-cycloserine is administered at a dose of about 10 mg/kg/d and is formulated to produce sustained blood levels in excess of about 25 microgram/mL.

### CROSS REFERENCE TO RELATED APPLICATIONS

This is a Continuation of U.S. patent application Ser. No. 16/166,101 which is a Continuation-in-Part of U.S. patent application Ser. No. 15/650,912, filed Jul. 16, 2017, now issued as U.S. Pat. No. 10,660,887, which is Continuation of U.S. patent application Ser. No. 13/936,198, filed Jul. 7, 2013, now issued as U.S. Pat. No. 9,737,531, which in turn claimed the benefit of the filing dates of U.S. Provisional Patent Application Nos. 61/741,114 and 61/741,115, both of which were filed on Jul. 12, 2012. This application also is a Continuation-in-Part of U.S. patent application Ser. No. 15/987,932, filed May 24, 2018, now issued as U.S. Pat. No. 10,583,138, which claims the benefit of the filing date of U.S. Provisional Patent Application No. 62/518,020, filed Jun. 12, 2017. This application is further a Continuation-in-Part of U.S. patent application Ser. No. 15/723,391, filed Oct. 3, 2017, which is the Continuation of U.S. patent application Ser. No. 13/982,460, filed Sep. 29, 2013, now issued as U.S. Pat. No. 9,789,093, which was the US National Stage of International Patent Application No. PCT/IL2012/050034, filed Jan. 30, 2012, and which in turned claimed the benefit of the filing date of U.S. Provisional Patent Application No. 61/437,700, filed Jan. 31, 2011, and 61/494,907, filed Jun. 9, 2011. The contents of the foregoing patent applications are incorporated by reference herein in their entireties.

### FIELD

This application relates to combination compositions for use in treatment of depression, and which can simultaneously alleviate the anxiogenic side effects of certain antidepressant and antipsychotic medications, thereby enabling continued and improved antidepressant and antipsychotic treatment. Methods for treatment of depression while simultaneously reducing medicament side effects, particularly anxiety, akathisia, and associated suicidality are also described herein.

### BACKGROUND

Major depression is a clinical syndrome that includes a persistent sad mood or loss of interest in activities, which persists for at least two weeks in the absence of treatment. Symptoms of major depression are typically measured using rating scales such as the Hamilton Depression Rating Scale (HAM-D) or the Beck Depression Inventory (BDI). In addition to including symptoms relevant to depressed mood, the HAM-D also contains symptoms sensitive to psychosis, including items for guilt, depersonalization/derealization and paranoia.

Major depression may also be associated with symptoms of anxiety, which may be measured with rating scales such as the Hamilton Rating Scale for Anxiety (HAM-A). Depressive disorders are divided in major depression (MDD) and bipolar depression (BPD), which may be diagnosed using criteria set forth in the Diagnostic and Statistical Manual, 5th edition, published by the American Psychiatric Association (DSM-5), which provides as well additional description of mental disorders. Major depression may also occur with and without melancholic features. In addition, depressive symptoms may occur in the context of anxiety disorders such as generalized anxiety disorder, dissociative disorders, personality disorders or adjustment disorders with depressed mood (DSM-5).

Other forms of depression include atypical depression, agitated depression, depression with mixed emotional features, cyclothymia, dysthymia minor depression and adjustment disorder with depressed mood. Bipolar depression may be divided into Bipolar I and Bipolar II subtypes based upon presence or absence of manic episodes. In bipolar disorder, depressive symptoms can occur in the context of either a depressive episode, or a mixed state in which symptoms of mania and depression occur simultaneously or in rapid sequence. Rapid cycling between mania and depressive episodes may also occur in some individuals.

N-methyl-D-aspartate receptors (NMDAR) are a type of receptor for the brain neurotransmitter glutamate. NMDAR participate in a range of brain functions including sensory processing, cognition, and emotion regulation.

NMDAR are comprised of multiple subunits termed GluN1, GluN2 and GluN3 (formerly NR1, NR2, NR3). Multiple forms of GluN1, GluN2 and GluN3 exist. In particular, GluN2 subunits are divided into GluN2A-D subforms, which are also termed NR2A-D subunits. NMDAR may consist of various combinations of GluN1, GluN2 and GluN3 subunits in various amounts. Agonists and antagonists may affect all NMDAR equivalently, or may be selective for NMDAR containing specific subunit types.

NMDAR contain binding sites for both the neurotransmitter glutamate and for the endogenous modulatory amino acids glycine and D-serine. The glutamate binding site also selectively binds the synthetic glutamate derivative N-methyl-D-aspartate with high affinity. This site is alternately referred to as the glutamate recognition site of the NMDA recognition site of the NMDAR.

The glycine/D-serine binding site has been referred to as the glycine modulatory site, the allosteric modulatory site or the glycine-B receptor. NMDAR form an ion channel that is blocked by several drugs of abuse, such as phencyclidine (PCP), ketamine or dizocilpine (MK-801). These compounds bind to a site that has been termed the PCP receptor. Agents that block the NMDAR-associated ion channel are collectively termed noncompetitive NMDAR antagonists, or NMDAR channel blockers. Blockade of NMDAR by channel blockers leads to a clinical psychotic state that closely resembles schizophrenia.

NMDAR may also be inhibited by antagonists that bind to the glutamate recognition site, the glycine recognition site, or the channel binding site.

NMDAR may also be inhibited by antagonists that bind to the glycine recognition site. Antagonists at the glycine recognition site may be full antagonists, which monotonically reduce NMDAR activity with increasing doses, or mixed agonists/antagonists, which differentially affect NMDAR function within different dose ranges. In specific, mixed agonists/antagonists may serve as agonists at lower doses, and as antagonists at higher doses. Partial agonists are compounds that may serve as agonists in the absence of full agonists, but as antagonists in the presence of full agonists.

Treatment-refractory depression (TRD) refers to a form of depression that responds poorly to currently available treatments (e g, nimh.nih.gov/trials/practical/stard/index.shtml June 2011) and which may have different underlying etiopathological mechanisms compared with other forms of depression. Combinations of antidepressants have not been shown to be superior to monotherapy for refractory depression and typically increase risk of side effects, and so are not recommended.

Current treatments for major depression consist primarily of older antidepressants, such as monoamine oxidase inhibitors (MAOI) and tricyclic antidepressants (TCAs) (e.g. imipramine, amitryptiline, desipramine, clomipramine) that were first developed in the 1960's, and newer agents such as tetracyclic antidepressants (TeCAs), (e.g. amoxapine, setiptiline, maprotiline, mianserin, mirtazapine), selective serotonin reuptake inhibitors (SSRIs) (e.g. sertraline, fluoxetine, citalopram, escitalopram, proxetine, fluvoxamine, trazadone) and serotonin/norephinephrine (SNRI) reuptake inhibitors (e.g., venlafaxine, desvenlafaxine, duloxetine). These agents work by modulating brain levels of monoamines such as norepinephrine and serotonin, and/or by blocking 5-HT2A receptors. MAOIs and TCAs are considered “broader spectrum” agents than SSRIs/SNRIs that were developed subsequently. MAOI, TCAs, TeCAs, SSRIs, SNRIs may collectively be considered traditional antidepressants.

TCAs and SSRIs show approximately equal efficacy in treatment of non-melanchoic forms of depression, suggesting overlapping but differentiable mechanisms of action. TCAs as a group show limited antipsychotic activity, alone or in combination with antipsychotics, but may be effective in treating persistent depressive symptoms in stabilized schizophrenia patients. TCAs have been shown to worsen psychosis in acutely decompensated schizophrenia patients, but to be relatively without effect on psychosis during the chronic phase of illness. In contrast, SSRIs and TeCAs may improve psychotic symptoms in addition to treatment of depression in refractory schizophrenia, suggesting a differential mechanism of action and mild antipsychotic potency.

Both TCAs and MAOIs have significant side effects that limit their use. In addition, TCAs may cause fatal arrhythmias following intentional or accidental overdose. At present, therefore, SSRIs and SNRIs represent the primary agents used for treatment of depression.

Other potential medications for depression include norepinephrine-dopamine reuptake inhibitors (NDRIs, also called norepinephrine reuptake inhibitors, NRI) such as bupropion or methylphenidate, and newer, atypical antidepressants with novel mechanisms of action, including vilazodone, vortioxetine, milnacipran and levomilnacipran.

Vilazodone is considered an atypical antidepressant in that it functions as both an SSRI and a partial agonist at 5HT1A receptors and has thus been termed a serotonin partial agonist and reuptake inhibitor (SPARI) (Schwartz et al., Vilazodone: A Brief Pharmacological and Clinical Review of the Novel Serotonin Partial Agonist and Reuptake Inhibitor, Ther Adv Psychopharmacol. 1:81-87, 2011). The combination of serotonin reuptake inhibition and 5-HT1A agonist is believed to function as an antidepressant at lower levels of serotonin transporter occupancy than otherwise (Kohler et al., J Psychopharmacogy, 30:13-22, 2016).

Vortioxetine is considered a multimodal antidepressant in that it functions as 1) a serotonin transport inhibitor, 2) a partial agonist at 5-HT1A receptors, and 3) a partial antagonist of 5-HT1B, 5HT1D and 5-HT7 receptors (Stahl S M, Modes and nodes explain the mechanism of action of vortioxetine, a multimodal agent (MMA): enhancing serotonin release by combining serotonin (5HT) transporter inhibition with actions at 5HT receptors (5HT1A, 5HT1B, 5HT1D, 5HT7 receptors) CNS Spectrums (2015), 20, 93-97)

Levopmilnacipran and milnacipran, while considered SNRIs, have a much lower ratio of serotonin:norephinephrine ratio than previously marketed SNRI agents. Thus, whereas venlafaxine, duloxetine, and desvenlafaxine all have serotonin:noepinephrine ratios of 10:1 or greater, levomilnacipran has a ratio of only 1.2:1 and milnacipran has a ratio of only 1.6:1 (Sansone RA, Sansone LA Serotonin norepinephrine reuptake inhibitors: a pharmacological comparison. Innov Clin Neurosci. 11 (3-4): 37-42). The mechanisms of action of milnacipran and levomilnacipran are thus more similar to TCAs than to other SNRIs, although they are safer than TCAs and do not share liability for fatal arrhythmia.

Atypical antipsychotics, especially those with significant antagonism at 5-HT2A receptors, may also be effective in treatment of either MDD or BPD. Atypical antipsychotics approved for treatment of MDD by the US FDA include aripiprazole, olanzapine, quetiapine XR and brexpiprazole. Atypical antipsychotics approved by the US FDA for the treatment of BPD include quetiapine, olanzapine/fluoxetine fixed dose combination, and lurasidone. Other atypical antipsychotics that target 5-HT2A receptors, including clozapine, ziprasidone, risperidone, asenapine, iloperidone, paliperidone, and cariprazine may also be effective in treatment of depression including mixed states of BPD.

Approved dosing levels for anti-depressants and anti-psychotics can be determined from standard sources, such as package inserts approved by the US Food and Drug Administration (FDA).

Risk for suicide is significantly increased in depressive disorders, but may respond differentially to medication versus depressive symptoms as a whole. While the risk of suicide increases in subjects with a depressive disorder, medications used to date to typically treat depressive disorders may also paradoxically increase suicidal tendencies.

Despite the wide range of pharmacological options, current treatment approaches for depression have severe limitations. Only 60-65% of patients respond to the initial treatment regimen and among those responding, fewer than half either reach remission or become symptom-free. Individuals not responding to a first course of antidepressant treatment are often switched to a different drug, with results that are generally modest and incremental.

Within major depression, treatment-refractory depression refers to a form of depression that responds poorly to currently available treatments (e.g., http://www.nimh.nih.gov/trials/practical/stard/index.shtml June 2011) and which may have different underlying etiopathological mechanisms compared with other forms of depression. Combinations of antidepressants have not been shown to be superior to monotherapy for refractory depression and typically increase risk of side effects and are not recommended.

D-cycloserine was first shown to induce significant anti-depressant effects in humans in 1959 when used at doses of 500-2000 mg/day (e.g. Crane G E. Cycloserine as an anti-depressant agent. Am J Psychiatry. 1959;115:1025-1029; Crane G E. The psychotropic effects of cycloserine: a new use for an antibiotic. Compr Psychiatry. 1961; 2:51-59). However, use of D-cycloserine at these doses was associated with significant psychotomimetic side effects including symptoms of tension, overactivity, psychotic reactions (e.g. paranoid symptoms) and depersonalization.

In one priority document for the present application (U.S. Pat. No. 9,789,093), it was shown that D-cycloserine used in TRD at a dose of 500-1000 mg/day in combination with antidepressant medications produced highly significant, large effect size improvement in depression, but did not produce significant psychotomimetic side effects. The lack of psychotomimetic side effects was unexpected over the prior literature and supported the novel utility of combined D-cycloserine and antidepressant medication.

Antidepressant medications used in the prior study included TCAs (desipramine, clomipramine, imipramine), TeCAs (mirtazapine, mianserin), SSRIs (escitalopram, fluoxetine, paroxetine, older SNRI (duloxetine, venlafaxine). A small-moderate effect size worsening in paranoia was observed (d=−0.31), suggesting a degree of residual psychotomimetic activity, but the effect did not reach statistical significance (p=0.12).

Despite showing unexpected benefit in depression, no pharmacokinetic studies were performed to determine D-cycloserine blood levels associated with clinical response.

In U.S. Pat. No. 9,486,453 it was demonstrated that that D-cycloserine unexpectedly reduced akathisia associated with 5-HT2A receptor antagonist and atypical antipsychotic treatment, when used at doses that produced NMDA receptor antagonistic blood plasma concentration measured at great then 25 micrograms/mL. Those studies did not, however, investigate effects of D-cycloserine on depressive symptoms. In U.S. Pat. No. 9,737,531, it was also demonstrated that other NMDA receptor antagonists such as D-CPPene were effective for reduction of akathisia in combination with 5-HT2A antagonists. It was further demonstrated that antidepressant agents (increased duloxetine, venlafaxine, mirtazapine) increased distance traveled in the elevated plus maze versus D-cycloserine 300 mg/kg alone, suggesting potential combined therapeutic benefit.

### SUMMARY

Described herein are methods for the treatment of humans suffering from depression, by administration of the inventive compositions in antidepression effective amounts.

Provided here is the first demonstration that D-cycloserine produces significant antidepressant effects in rodents specifically at D-cycloserine doses of 300 mg/kg or above. Furthermore, provided is the first demonstrate that doses associated with significant antidepressant response in the forced swim test produce blood D-cycloserine levels in excess of 25 micrograms/mL, suggesting that anti-depressant effects of D-cycloserine are specifically related to its actions as an NMDA receptor antagonist.

In humans, it is known that doses of 10 mg/kg or above produce blood levels in excess of 25 microgram/mL. Provided herein is the first demonstration that antidepressant effects of D-cycloserine in humans will occur at D-cycloserine doses of 10 mg/kg or above, that produce blood levels in excess of 25 microgram/mL.

In one embodiment, this invention provides an oral or parenteral dosage regimen that includes two active therapeutic agents, wherein the first agent is an NMDAR antagonist, and the second active agent is an antidepressant.

In some embodiments, the antidepressant is drawn from a list that includes a tetracyclic antidepressant (TeCA), selective serotonin reuptake inhibitor (SSRI), a serotonin/norephinephrine reuptake inhibitor (SNRI), a norepinephrine reuptake inhibitor (NRI), a tricyclic antidepressant, an monoamine oxidase inhibitor (MAOI) or a combination thereof.

NMDAR antagonists may be drawn from antagonists at the glycine, glutamate or polyamine recognition sites.

NMDAR antagonists may be non-selective antagonists or selective antagonists at NMDAR containing specific subunits such as the NR2A or NR2B subunits.

In some embodiments, the NMDAR antagonist is drawn from a list that includes ketamine, dextromethorphan, GlyX-13, NRX-1074, CNS-1102, AZD6765, or CGS-19755.

In a preferred embodiment of the invention, the NMDAR antagonist is D-cycloserine, which in certain embodiments is administered at a NMDAR antagonist dosage of greater than 500 mg per day but less than 1000 mg per day.

In some embodiments, the NMDAR antagonist is D-cycloserine, administered at a dose that produces peak blood levels in excess of 25 microgram/mL, but less than 125 microgram/mL.

In some embodiments, the NMDAR antagonist is D-cycloserine, administered at a dose that produces sustained blood levels in excess of 25 microgram/mL, but less than 125 microgram/mL. In some embodiments, these blood levels are sustained for a duration of 12 hours following D-cycloserine administration.

In some embodiments, this invention provides a method for treatment of a psychosis in a subject in need thereof, by providing the subject with an oral or parenteral dosage regimen as herein described.

In some embodiments this invention provides a method for treatment of depression in a subject in need thereof, by providing said subject with an oral or parenteral dosage regimen as herein described

In some embodiments, the subject suffers from mania, or in some embodiments, the subject suffers from bipolar disorder, which may be bipolar I disorder or bipolar II disorder

In some embodiments, this invention provides a method for treating symptoms of autism or autism spectrum disorder in a subject in need thereof, by providing said subject with an oral or parenteral dosage regimen as herein described.

In some embodiments, the invention provides a method for reducing side effects associated with antipsychotic medications to a subject in need of such treatment by providing the subject with an oral or parenteral dosage regimen as herein described.

In some embodiments, this invention provides a method for reducing side effects associated with antidepressant medications to a subject in need of such treatment, by providing said subject with an oral or parenteral dosage regimen as herein described. The reduction of such side effects provides an unexpected and superior treatment for depression, particularly allowing for continued administration of the antidepressant agent. For example, the combination therapies provided herein, such as between DCS and lurasidone, can allow for continued treatment with lurasidone, but with decreased suicide ideation, thereby extending and increasing the efficacy of the treatment.

In some embodiments, this invention provides a method for reducing side effects associated with NMDAR antagonist medications to a subject in need of such treatment, by providing the subject with an oral or parenteral dosage regimen as herein described.

In some embodiments, a gelling agent such as hydroxypropyl methylcellulose, together with one or more pharmaceutically acceptable excipients is used for manufacture of the sustained release agent.

In some embodiments, the sustained release formulation includes a hydrophilic matrix comprising a gelling agent, such as hydroxypropyl methylcellulose, an NMDAR antagonist, an antidepressant and pharmaceutically acceptable salts thereof, together with one or more pharmaceutically acceptable excipients.

In some embodiments, the sustained release formulation reduces gastric degradation of the NMDAR antagonist or the antidepressant agent by protecting the NMDAR antagonist from hydrolysis.

In some embodiments, a covalent modification is made to D-cycloserine to reduce its susceptibility to hydrolysis and gastric degradation. In some embodiments, the covalent modification creates a prodrug that is resistant to hydrolysis and gastric degradation. In some embodiments, the covalent modification is made to the primary amide group of D-cycloserine. In some embodiments, the covalent modification is made to the secondary amide group of the D-cycloserine molecule. In some embodiments, the covalent modification consists of addition of a methyl group. In some embodiments, the covalent modification consists of addition of a fully or partially-hydrogenated polyalkyl moiety. In some embodiments, the covalent modification creates a D-cycloserine prodrug that is metabolized by the body to D-cycloserine.

In some embodiments both the NMDAR antagonist and the antidepressant or antipsychotic medication are manufactured for sustained release in common.

In some embodiments, the NMDAR antagonist is manufactured for sustained release, and combined with an antidepressant or antipsychotic agent

In some embodiments, the antidepressant or antipsychotic agent is manufactured for sustained release, and combined with an NMDAR antagonist.

In one embodiment, an NMDAR antagonist and an antidepressant or antipsychotic agent are selected for release characteristics permitting once daily dosing of the combined medicament, and would not require separate sustained release manufacture.

The foregoing and other objects, features, and advantages will become more apparent from the following detailed description, which proceeds with reference to the accompanying figures.

### BRIEF DESCRIPTION OF THE DRAWINGS

FIG. 1 is a graph showing the effect of indicated mg/kg (mpk) doses of D-cycloserine (DCS) on immobility time in the murine forced swim test (FST) following oral (PO) administration relative to no-drug negative control (“Ctl”) or active comparator (sertraline, “Sert”). *p<0.05 vs. control. ***p<0.001 vs. control.

FIG. 2 is a graph showing the effect of DCS in combination with antidepression agents as indicated on immobility time in the FST. As shown, *p<0.05, **p<0.01, ***p<0.001 No DCS vs. 300 mg/kg. #p<0.05 vs. DCS 300 mg/kg alone.

FIG. 3 is a graph showing the effect of DCS in combination with antidepression agents as indicated on immobility time in the FST. Control and test compounds are abbreviated as in the description of FIG. 1. ***p<0.001 vs. control.

FIG. 4: is a graph showing the effect of DCS in combination with atypical antipsychotics as indicated in the FST. “Ctl.”, control (no antipsychotic); “Olz 0.6”, olanzipine at 0.6 mg/kg; “Risp 0.1”, risperidone at 0.1 mg/kg; “Aripip 1”, aripiprazole at 1 mg/kg; “Qtp 10”, quetiapine at 10 mg/kg; “Brexpip 0.3”, brexpiprazole at 0.3 mg/kg. *p<0.05 vs. between indicated conditions ***p<0.001 between indicated conditions.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Similarly, use of the word “about” in front of a numerical value or range indicates that values on either side, typically ±10%, of the stated value or range can be considered to fall within the scope of that value or range. Although methods and materials similar or equivalent to those described herein can be used in the practice or testing of this disclosure, suitable methods and materials are described below. The term “comprises” means “includes.” “Consisting essentially of” indicates a composition, method, or process that includes only those listed features as the active or essential elements, but can include non-active elements in addition. The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

As used herein, reference to an “effective” amount or a “therapeutically effective amount” of therapeutic agents referenced herein, it is meant a nontoxic but sufficient amount of the same to provide the desired effect. In a combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual.

D-cycloserine, or DCS, refers to the chemical D-cycloserine (CA Index Name: 3-Isoxazolidinone, 4-amino-, (4R)-(9CI); CAS Registry No. 68-41-7), or pharmaceutically acceptable salts thereof. DCS is an FDA (United States Food and Drug Administration)-approved drug for treatment of tuberculosis, and is sold by Eli Lilly and Company under the trade name Seromycin®. DCS is a structural analog of D-alanine, and is a broad-spectrum antibiotic produced by some strains of Streptomyces orchidaceus and S. garphalus.

### II. Methods for Treatment of Depression

As shown herein, D-cycloserine induces anti-depressant effects in rodents selectively and unexpectedly only at doses that produce sustained blood (e.g. serum or plasma) levels of >25 microgram/mL. These findings complement the previous demonstration (U.S. Pat. No. 9,789,093) that antidepressants unexpectedly prevent psychotic symptoms associated with NMDAR antagonist usage, but which did not indicate optimal dosing levels or demonstrate as discussed herein that NMDAR antagonists in combination with antidepressant medications can decrease the side effect of the antidepressants.

Moreover, it is unexpectedly shown herein that manipulations reducing gastric degradation of D-cycloserine may lead to increased blood levels relative to the dose administered.

In some embodiments, the described compositions provide an oral or parenteral dosage regimen consisting essentially of two active ingredients, wherein a first of said ingredients is an NMDAR receptor antagonist, and the second active ingredient is an antipsychotic or antidepressant agent. In some embodiments, according to this aspect, the antipsychotic or antidepressant agent comprises any such agent as herein described and others that are known in the art, for example, a tetracyclic antidepressant (TeCA), selective serotonin reuptake inhibitor (SSRI), a serotonin/norephinephrine reuptake inhibitor (SNRI), a norepinephrine uptake inhibitor (NRI), a norepinephrine-dopamine reuptake inhibitor (NDRI), a selective 5-HT2A receptor antagonist, a selective 5-HT2A receptor inverse agonist, an atypical antidepressant, or an antipsychotic approved for use in treatment of depression or a combination thereof.

The compositions for use in the methods described herein include an NMDAR receptor antagonist. N-methyl-D-aspartate receptors (NMDAR) are a type of receptor for the brain neurotransmitter glutamate. NMDAR participate in a range of brain functions including sensory processing, cognition, and emotion regulation.

NMDAR are comprised of multiple subunits termed GluN1, GluN2 and GluN3 (formerly NR1, NR2, NR3). Multiple forms of GluN1, GluN2 and GluN3 exist. In particular, GluN2 subunits are divided into GluN2A-D subforms, which are also termed NR2A-D subunits. NMDAR may consist of various combinations of GluN1, GluN2 and GluN3 subunits in various amounts. Agonists and antagonists may affect all NMDAR equivalently, or may be selective for NMDAR containing specific subunit types.

NMDAR contain binding sites for both the neurotransmitter glutamate and for the endogenous modulatory amino acids glycine and D-serine.

The glutamate binding site also selectively binds the synthetic glutamate derivative N-methyl-D-aspartate (NMDA) with high affinity. This site is alternately referred to as the glutamate recognition site of the NMDA recognition site of the NMDAR.

The glycine/D-serine binding site has been referred to as the glycine modulatory site, the allosteric modulatory site or the glycine-B receptor.

NMDAR form an ion channel that is blocked by several drugs of abuse, such as phencyclidine (PCP), ketamine or dizocilpine (MK-801). These compounds bind to a site that has been termed the PCP receptor. Agents that block the NMDAR-associated ion channel are collectively termed non-competitive NMDAR antagonists, or NMDAR channel blockers. Blockade of NMDAR by channel blockers leads to a clinical psychotic state that closely resembles schizophrenia.

Other compounds that block NMDAR via the channel site include AZD6765 (Lanicemine, AstraZeneca)

Other NMDAR antagonists are disclosed in U.S. Pat. No. 9,512,133, which is incorporated by reference herein in its entirety.

Low affinity NMDAR antagonists, such as memantine, may be distinguished from high affinity antagonists such as PCP, ketamine or dizocilpine. In general, low affinity NMDAR antagonists do not induce schizophrenia-like psychosis or PCP-like behavioral effects in rodents.

NMDAR may also be inhibited by antagonists that bind to the glutamate recognition sites, the glycine recognition site, or the polyamine (redox sensitive) binding site.

Agents that block binding of the endogenous amino acids glutamate or glycine to their respective binding sites and which have no intrinsic activity are termed competitive glutamate or glycine site antagonists. Agents which bind to these sites but which have intrinsic activity lower the endogenous ligands are termed partial agonists and may function as mixed agonists/antagonists.

Selfotel (CGS19755) is an example of an antagonist that binds to the glutamate recognition site. Several such compounds were developed for CNS indications such as stroke or epilepsy. When used at doses sufficient to significantly inhibit NMDAR, these compounds, like channel blockers, lead to clinical psychotomimetic symptoms.

Additional compounds that functions as antagonists of the glutamate recognition site include aptiganel (Cerestat, CNS-1102) and related compounds as disclosed in Reddy et al., J Med Chem 37:260-7. 1994.

Additional compounds that function as antagonists of the glutamate recognition site include alpha.-amino-carboxylic acid and phosphonic acid functionalities separated by a variety of spacer units. An unembellished example is 2-amino-5-phosphonovaleric acid (AP5) (Watkins, J. C.; Evans, R. H., Annu. Rev. Pharmacol. Toxicol. 1981, 21, 165), which contains a saturated carbon chain. More complex examples, which contain elements enhancing structural rigidity and therefore potency, include CPP, cis-4-(phosphonomethyl)-2-piperidinecarboxylic acid (CGS-19755) (Lehman, J. et al., J. Pharmacol. Exp. Ther. 1988, 246, 65), and (E)-2-amino-4-methyl-5-phosphono-3-pentenoic acid (CGP-37849) (Schmutz, M. et al., Abs. Soc. Neurosci. 1988, 14, 864). See U.S. Pat. No. 7,345,032, issued Mar. 18, 2008 and U.S. Pat. No 5,168,103, which are incorporated herein by reference in their entireties.

Non-limiting examples of NMDAR antagonists for use in the described compositions and methods include ketamine, Selfotel, aptiganel, CPP, CGP-37849, felbamate, Gavestinel N-(6,7-dichloro-2,3-dioxo-1,2,3,4-tetrahydro-quinoxalin-5-yl)-N-(2-hydroxy-ethyl)-methanesulfonamide and 6,7-dichloro-5-[3-methoxymethyl-5-(1-oxypyridin-3-yl)-[1,2,4]triazol-4-yl]-1,4-dihydro-quinoxa-line-2,3-dione, 4-(3-phosphono-propyl)-piperazine-2-carboxylic acid (CPP), D-(E)-4-(3-phosphonoprop-2-enyl)piperazine-2-carboxylic acid (D-CPPene), SDZ-220581, PD-134705, LY-274614 and WAY-126090; quinolinic acids, such as kynurenic acid, 7-chloro-kynurenic acid, 7-chloro-thiokynurenic acid and 5,7-dichloro-kynurenic acid, prodrugs thereof, such as 4-chlorokynurenine and 3-hydroxy-kynurenine; 4-aminotetrahydrochinolin-carboxylates, such as L-689,560; 4-hydroxyquinolin-2(1H)-ones, such as L-701,324; quinoxalinediones, such as licostinel (ACEA-1021) and CGP-68,730A; 4,6-dichloro-indole-2-carboxylate derivatives such as MDL-105,519, gavestinel (GV-150,526) and GV-196,771A; tricyclic compounds, such as ZD-9,379 and MRZ-2/576, (+)-HA-966, morphinan derivatives such as dextromethorphan and dextrophan; benzomorphans, such as BIII-277CL; other opioids, such as dextropropoxyphene, ketobemidone, dextromethadone and D-morphine; amino-adamantanes, such as amantadine and memantine; amino-alkyl-cyclohexanes, such as MRZ-2/579; ifenprodil and ifenprodile-like compounds such as eliprodil and PD-196,860; iminopyrimidines; or other NMDA-antagonists such as nitroprusside, D-cycloserine, 1-aminocyclopropane-carboxylic acid, dizocilpine (MK 801) and its analogs, (R)-ketamine, (S)-ketamine, remacemide and its des-glycinyl-metabolite FPL-12,495, AR-R-15,896, methadone, sulfazocine, AN19/AVex-144, AN2/AVex-73, Besonprodil, CGX-1007, EAB-318, and NPS-1407.

NMDAR may also be inhibited by antagonists that bind to the glycine recognition site such as D-cycloserine.

D-cycloserine is a compound that acts as both a partial agonist and mixed agonist/antagonist at the glycine modulatory site of the NMDA receptor. Levels of D-cycloserine in blood (e.g. plasma or serum) can be assessed using standard chromatographic results including without limitation high performance liquid chromatography (HPLC). D-cycloserine levels, in general, are equivalent whether serum or plasma are used for determination.

In in vitro studies, D-cycloserine acts as NMDA receptor agonist at doses of up to approximately 100 μM (also expressed as 10.2 microgram/mL based on a MW for D-cycloserine of 102 g/mole) (Watson G B, Bolanowski M A, Baganoff M P, Deppeler C L, Lanthorn T H. D-cycloserine acts as a partial agonist at the glycine modulatory site of the NMDA receptor expressed in Xenopus oocytes. Brain Res. 1990; 510(1):158-160; Hood W F, Compton R P, Monahan J B. D-cycloserine: a ligand for the N-methyl-D-aspartate coupled glycine receptor has partial agonist characteristics. Neurosci Lett. 1989; 98(1):91-95), with half-maximal effects at approximately 10 μM.

A more recent study has shown a decline in agonist effects of D-cycloserine in vitro between doses of 10 and 100 μM (Moskal J R, Kuo A G, Weiss C, et al. GLYX-13: a monoclonal antibody-derived peptide that acts as an N-methyl-D-aspartate receptor modulator. Neuropharmacology. 2005; 49(7):1077-1087) suggestive of antagonist effects at concentrations above 100 μM. The preferred blood D-cycloserine concentration for treatment of depression of about 25 microgram/mL or above corresponds to a molar concentration of about 245 μM and thus falls within the antagonist range.

In rodents, agonist effects of D-cycloserine have been reported with doses of 5-10 mg/kg (U.S. Pat. No. 4,904,681; Emmett M R, Mick S J, Cler J A, Rao T S, Iyengar S, Wood P L. Actions of D-cycloserine at the N-methyl-D-aspartate-associated glycine receptor site in vivo. Neuropharmacology. 1991; 30(11):1167-1171). In one study (Balla A, Schneider S, Sershen H, Javitt D C. Effects of novel, high affinity glycine transport inhibitors on frontostriatal dopamine release in a rodent model of schizophrenia. Eur Neuropsychopharmacol. 2012; 22(12):902-910) an NMDA receptor agonist dose of 30 mg/kg was shown to produce plasma D-cycloserine levels of approximately 10 microgram/mL, and brain D-cycloserine levels of approximately 6 microgram/mL. In general, rodent doses associated with NMDA receptor agonist effects are in the range of about 5-50 mg/kg.

Apparent NMDA receptor antagonist effects in rodents have been reported with doses in the range of about 80-320 mg/kg in rodents. For example, in one study of kainic acid-induced seizures in rats, DCS doses of 80-320 mg/kg produced inhibition of seizure activity equivalent to that observed with the NMDAR antagonist MK-801 (Baran H, Loscher W, Mevissen M (1994) The glycine/NMDA receptor partial agonist D-cycloserine blocks kainate-induced seizures in rats. Comparison with MK-801 and diazepam. Brain Res 652: 195-200). However, blood or brain D-cycloserine levels associated with these effects were not evaluated, nor were effects in rodent models of depression.

D-cycloserine is commonly supplied in 250 mg capsules, permitting dosing at 250 mg, 500 mg, 750 mg or 1000 mg per day. D-cycloserine may also be administered at intermediate doses, for example, 200 mg, 300 mg, 400 mg, 500 mg, 600 mg, 700 mg, 800 mg, 850 mg, 900 mg or 950 mg per day in order to induce the required plasma concentration of about 25 micrograms/mL.

In a particular embodiment, the D-cycloserine component of the described compositions is administered at a dosage of greater than about 500 mg per day but less than about 1000 mg per day, to produce a net NMDAR antagonist effect wherein sustained serum levels are in excess of about 25 micrograms/mL, but less than about 125 micrograms/mL. Particular embodiments of the described compositions include a D-cycloserine component administered at a dosage that produce sustained serum levels of about 25 to about 40 micrograms/mL, such as about 26, about 27, about 28, about 29, about 30, about 31, about 32, about 33, about 34 and about 35 micrograms/mL or greater.

Pharmacokinetics (PK) of D-cycloserine in humans after a dose of 500 mg have been previously studied. Critical parameters when examining the PK of a drug in blood include maximum (peak) concentration achieved (Cmax), time to maximum concentration (Tmax) and area under the curve (AUC) during the dosing interval.

For example, Zhu et al. (Zhu M, Nix D E, Adam R D, Childs J M, Peloquin C A. Pharmacokinetics of cycloserine under fasting conditions and with high-fat meal, orange juice, and antacids. Pharmacotherapy. 2001; 21(8):891-7) showed median Cmax values of 14.8 microgram/mL following administration of a single D-cycloserine dose of 500 mg under fasting conditions, with a range of 12.1-30.6 microgram/mL. Median AUC levels over 24 hr were 214 microgram-hr/mL with a range of 163-352, corresponding to median sustained plasma levels of 8.9 microgram/mL with a range of 6.8-14.7 microgram/mL.

Park et al., (Park S I, Oh J, Jang K, Yoon J, Moon S J, Park J S, Lee J H, Song J, Jang I J, Yu K S, Chung J Y. Pharmacokinetics of Second-Line Antituberculosis Drugs after Multiple Administrations in Healthy Volunteers. Antimicrob Agents Chemother. 2015; 59(8):4429-35) evaluated pharmacokinetics of 250 mg PO D-cycloserine given every 12 hrs, and observed mean Cmax values of 24.9 microgram/mL and a mean AUC over 12 hrs of 242.3 mg-h/L, corresponding to a mean plasma level of 20.2 microgram/mL.

Hung et al., 2014 (Hung W Y, Yu M C, Chiang Y C, Chang J H, Chiang C Y, Chang C C, Chuang H C, Bai K J. Serum concentrations of cycloserine and outcome of multidrug-resistant tuberculosis in Northern Taiwan. Int J Tuberc Lung Dis. 2014; 18(5):601-6) evaluated PK levels during clinical treatment with DCS. Mean dose across subjects was 8.8 mg/kg, with the majority of subjects (n=27) receiving 500 mg/day DCS, and a minority either 750 mg/d (n=4) or 250 mg/d (n=2). DCS concentrations at 2 and 6 hr after dosing were 19.7 and 18.1 microgram/mL.

Hung et al., 2014 also evaluated dosing as a function of mg/kg, and found a highly significant correlation between serum concentration and D-cycloserine dose. Based upon this relationship, a human dose exceeding 10 mg/kg is required to produce a serum level in excess of 25 microgram/mL. This dose would translate into a daily dose of 700 mg/kg for an average 70 kg individual.

Thus, a consistent finding of human D-cycloserine PK studies is that sustained blood levels following administration of D-cycloserine at a dose of about 500 mg/day are consistently below about 25 microgram/mL, and so would not be expected to act as a net NMDAR antagonist as required for the described compositions and methods. By extension, to achieve the net NMDAR antagonist effect required in the compositions and methods described herein (greater than about 25 microgram/mL), administration of greater than about 500 mg/day DCS is required, with preferred daily doses of above about 700 mg/kg for the average individual, and preferred weight-based dosing of >about 10 mg/kg. The described compositions can include D-cycloserine provided at a weight-based dose of between about 10 to about 25 mg/kg/day, such as but not limited to about 12, about 14, about 15, about 16, about 20, about 22, and about 24 mg/kg/day.

In addition to the dosages described herein by the sustained blood concentration produced, the mg/kg/day, and the mg/day daily dose, dosages can also be represented in terms of molarity. Given the molecular weight of D-cycloserine as 102 g/mole, the proposed concentration of greater than about 25 micrograms/mL corresponds to a molarity-based level of greater than about 245 micromolar. As previously described, and as shown herein (see Table 2 for example), dosages below this concentration will not provide the net antagonistic effect utilized in the current compositions and methods.

The inclusion of an enteric coating is known to decrease sensitivity of medications to hydrolysis in the stomach. Reduction of hydrolysis by enteric coating would decrease the oral dose of D-cycloserine needed to achieve the desired NMDAR-antagonist blood levels of between about 25 and about 125 micrograms/mL, particularly between about 25 and about 40 micrograms/mL. Therefore, in particular embodiments wherein the pharmaceutical composition includes an enteric coating, the D-cycloserine can be provided at less than about 500 mg/day, such as about 450, about 400, about 350, about 300, about 250 mg/day or at an even lower amount. It will be appreciated that the dosage of D-cycloserine for use in the described compositions is one that produces a net NMDAR antagonist effect, corresponding to peak and sustained blood levels of between about 25 and 125 micrograms/mL. Additional approaches for protecting compounds from gastric degradation are disclosed in U.S. Pat. Nos. 8,105,626, 4,853,230, 6,328,994, US Patent Publication No. 2002/0039597 and Japanese patent publication No. JP62-277322A, which are hereby incorporated by reference.

Felbamate is another example of a NMDAR antagonist compound that may act via the glycine binding site. When administered to humans, felbamate produces psychotic effects that limit its clinical utility (e.g. Besag F M, Expert Opin Drug Saf 3:1-8, 2004).

Gavestinel (GV-150,526) is another example of a glycine binding site antagonist. Other compounds are disclosed in DiFabrio et al., J Med Chem 40:841-50, 1997, which is hereby incorporated by reference.

Glyx-13 (Naurex) and NRX-1059 (Naurex) are other examples of compounds that bind to the glycine binding site. Other compounds effective in blocking the glycine binding site are disclosed in U.S. Pat. Nos. 8,951,968, and 8,673,843, which are hereby incorporated by reference.

Other examples of glycine site antagonists that are suitable for use in the pharmaceutical compositions and methods of this invention are those referred to in the following: U.S. Pat. No. 6,667,317 which was issued on Dec. 23, 2003; U.S. Pat. No. 6,080,743 which was issued Jun. 27, 2000; U.S. Pat. No. 5,990,108, which was issued on Nov. 23, 1999; U.S. Pat. No. 5,942,540, which issued on Aug. 24, 1999; World Patent Application WO 99/34790 which was published on Jul. 15, 1999; WO 98/47878, which was published on Oct. 29, 1998; World Patent Application WO 98/42673, which was published on Oct. 1, 1998; European Patent Application EP 966475A1, which was published on Dec. 29, 1991; World Patent Application 98/39327, which was published on Sep. 11, 1998; World Patent Application WO 98/04556, which was published on Feb. 5, 1998; World Patent Application WO 97/37652, which was published on Oct. 16, 1997; U.S. Pat. No. 5,837,705, which was issued on Oct. 9, 1996; World Patent Application WO 97/20553, which was published on Jun. 12, 1997; U.S. Pat. No. 5,886,018, which was issued on Mar. 23, 1999; U.S. Pat. No. 5,801,183, which was issued on Sep. 1, 1998; World Patent Application WO 95/07887, which was published on Mar. 23, 1995; U.S. Pat. No. 5,686,461, which was issued on Nov. 11, 1997; U.S. Pat. No. 5,622,952, issued Apr. 22, 1997; U.S. Pat. No. 5,614,509, which was issued on Mar. 25, 1997; U.S. Pat. No. 5,510,367, which was issued on Apr. 23, 1996; European Patent Application 517,347A1, which was published on Dec. 9, 1992; U.S. Pat. No. 5,260,324, which published on Nov. 9, 1993. The foregoing patents and patent applications are incorporated herein by reference in their entireties.

Other examples of glycine site antagonists that can be used in the pharmaceutical composition and methods of this invention are N-(6,7-dichloro-2,3-dioxo-1,2,3,4-tetrahydro-quinoxalin-5-yl)-N-(2-hydroxy-ethyl)-methanesulfonamide and 6,7-dichloro-5-[3-methoxymethyl-5-(1-oxypyridin-3-yl)-[1,2,4]triazol-4-yl]-1,4-dihydro-quinoxa-line-2,3-dione.

Additional NMDAR antagonists are disclosed in Schiene et al., U.S. Patent Publication No. US2011/0306674, which is incorporated herein by reference in its entirety, and include without being limited thereto, N-containing phosphonic acids, such as norvaline (AP5), D-norvaline (D-AP5), 4-(3-phosphono-propyl)-piperazine-2-carboxylic acid (CPP), D-(E)-4-(3-phosphonoprop-2-enyl)piperazine-2-carboxylic acid (D-CPPene), cis-4-(phosphonomethyl)-2-piperidine carboxylic acid (Selfotel, CGS 19755), SDZ-220581, PD-134705, LY-274614 and WAY-126090; quinolinic acids, such as kynurenic acid, 7-chloro-kynurenic acid, 7-chloro-thiokynurenic acid and 5,7-dichloro-kynurenic acid, prodrugs thereof, such as 4-chlorokynurenine and 3-hydroxy-kynurenine; 4-aminotetrahydrochinolin-carboxylates, such as L-689,560; 4-hydroxyquinolin-2(1H)-ones, such as L-701,324; quinoxalinediones, such as licostinel (ACEA-1021) and CGP-68,730A; 4,6-dichloro-indole-2-carboxylate derivatives such as MDL-105,519, gavestinel (GV-150,526) and GV-196,771A; tricyclic compounds, such as ZD-9,379 and MRZ-2/576, (+)-HA-966, morphinan derivatives such as dextromethorphan and dextrophan; benzomorphans, such as BIII-277CL; other opioids, such as dextropropoxyphene, ketobemidone, dextromethadone and D-morphine; amino-adamantanes, such as amantadine and memantine; amino-alkyl-cyclohexanes, such as MRZ-2/579; ifenprodil and ifenprodile-like compounds such as eliprodil and PD-196,860; iminopyrimidines; or other NMDA-antagonists such as nitroprusside, D-cycloserine, 1-aminocyclopropane-carboxylic acid, dizocilpine (MK 801) and its analogs, phencyclidine (PCP), ketamine ((R,S)-2-(2-Chlorphenyl)-2-(methylamino)cyclohexan-1-on), (R)-ketamine, (S)-ketamine, remacemide and its des-glycinyl-metabolite FPL-12,495, AR-R-15,896, methadone, sulfazocine, AN19/AVex-144, AN2/AVex-73, Besonprodil, CGX-1007, EAB-318, Felbamate and NPS-1407. NMDA-Antagonists are, for example, disclosed in “Analgesics,” edited by H. Buschmann, T. Christoph, E. Friderichs, C. Maul, B. Sundermann, 2002, Wiley-VCH Verlag GmbH & Co. KGaA, Weinheim, Germany, in particular pages 389-428. The respective parts of the disclosure are hereby incorporated by reference and form part of the present disclosure.

Antagonists may be selective for the GluN2B (NR2B) containing subtype. Examples of compounds that are selective for NR2B containing receptors include ifenprodil, traxoprodil (CP-101,606), besonprodil, Ro25-6981, MK-0657 (also termed CERC-301, Rislenemdaz) and EVT-101.

Along with identified NMDAR antagonists, additional such compounds can be identified using well-validated electrophysiological assays such as modulation of NMDA-receptor mediated responses to NMDA glutamate-site agonists, or radioreceptor assays, such as modulation of binding to the NMDA PCP-receptor channel binding site. Glycine site agonists and antagonists can also be distinguished based upon both electrophysiology and receptor binding from compounds such as phencyclidine (PCP) or ketamine that bind to the channel site. Partial agonists are defined as compounds that have reduced efficacy for inducing conformational change in receptors (typically 40-80%) relative to full agonists, and which may induce agonist effects in the absence of a full agonist, but antagonist effects in the presence of a full agonist.

The NMDAR antagonist ketamine is currently approved as an anesthetic agent. It has also been reported to show beneficial effects in treatment resistant depression in small scale clinical trials. However, its utility is limited by psychotomimetic effects. Ketamine is a racemic mixture of S- and R-ketamine The S- and R-enantiomers of ketamine are also under separate investigational development for use in treatment-resistant depression. The low affinity NMDAR antagonist memantine is approved for use in dementia. Otherwise, NMDAR antagonists have no established clinical utility.

In general, NMDAR antagonists are considered contraindicated for use in schizophrenia or depression. For example, the NMDAR antagonist D-cycloserine is contraindicated by FDA for use in depression, severe anxiety or psychosis. Barlow (US Patent Publication No. 2010/0216805) has proposed that D-cycloserine may be effective in treating depression, but only at concentrations of below 100 micromolar, which would correspond to plasma concentrations of under 11 microgram/mL, in which D-cycloserine is acting as a net agonist, rather than the net antagonist activity of FDA contraindications and the current invention.

Plasma levels of D-cycloserine may be assessed using standard chromatographic techniques such as but not limited to high performance liquid chromatography. In general, blood levels above about 25 microgram/mL lead to increased risk in psychotomimetic effects

Anti-depressant effects of psychotropic compounds may be assessed using well-established rodent behavioral assays such as the forced swim test (e.g. Cryan et al., Neuroscience and biobehavioral reviews. 29:547-69, 2005).

D-cycloserine is a compound currently approved for treatment of tuberculosis (TB). Psychotropic effects of cycloserine were noted in the late 1950's in patients being treated for TB. In an initial report, effects of cycloserine were noted on symptoms such as anorexia, asthenia and insomnia (Crane G E. Cycloserine as an anti-depressant agent. Am J Psychiatry. 1959; 115:1025-1029). However, no formal psychiatric diagnoses were made. Furthermore, D-cycloserine was recommended primarily for treatments of tension and insomnia, as opposed to depression.

Because of its ability to bind to NMDAR and because of theories linking NMDAR to schizophrenia, D-cycloserine has been studied in treatment resistant schizophrenia. At low doses, D-cycloserine has been found to produce beneficial effects in some but not all studies, and may exacerbate symptoms in individuals receiving clozapine. Furthermore, at higher doses (>250 mg), D-cycloserine exacerbates psychosis and so according to package label insert is contra-indicated in psychosis, depression and anxiety disorders (Lilly. Seromycin (cycloserine) capsules prescribing information. Indianapolis, Ind.; 2005 Apr. 28).

D-cycloserine has also been assessed in the treatment of anxiety disorders, PTSD and enhancement of learning and memory at doses of 50-500 mg, with the goal primarily of enhancing NMDAR function (e.g. Goff D C. D-cycloserine in Schizophrenia: New Strategies for Improving Clinical Outcomes by Enhancing Plasticity. Curr Neuropharmacol. 2017; 15(1):21-34; Otto et al., Enhancement of Psychosocial Treatment With D-Cycloserine: Models, Moderators, and Future Directions. Biol Psychiatry. 2016; 80(4):274-83; Schade S, Paulus W. D-Cycloserine in Neuropsychiatric Diseases: A Systematic Review. Int J Neuropsychopharmacol. 2016; 19(4).

In addition, use of D-cycloserine has been disclosed for enhancement of cognition at doses of up to 100 mg and for treatment of a wide variety of neuropsychiatric disorders at doses of up to 500 mg (Tsai, U.S. Pat. No. 6,228,875.

It has also been disclosed that D-cycloserine may be useful in augmenting cognition in Alzheimer disease, Parkinson's disease and other dementias (McDevitt U.S. Pat. No. 9,877,951).

In both anxiety and schizophrenia studies, it has been noted that effects of D-cycloserine may decrease over time during repeated treatment, leading some to advocate use of weekly, rather than daily, D-cycloserine (Goff et al. Once-weekly D-cycloserine effects on negative symptoms and cognition in schizophrenia: an exploratory study. Schizophr Res. 2008; 106(2-3):320-7).

When used as augmentation of behavior therapy for anxiety, D-cycloserine is used episodically in combination with behavioral therapy sessions (Otto et al., Enhancement of Psychosocial Treatment With D-Cycloserine: Models, Moderators, and Future Directions. Biol Psychiatry. 2016; 80(4):274-83; Schade S, Paulus W. D-Cycloserine in Neuropsychiatric Diseases: A Systematic Review. Int J Neuropsychopharmacol. 2016; 19(4).).

Prior research with D-cycloserine in preclinical models has also not suggested its usefulness at high dose in treatment of depression (e.g. Papp M, Moryl E. Antidepressant-like effects of 1-aminocyclopropanecarboxylic acid and D-cycloserine in an animal model of depression. Eur J Pharmacol. 1996; 316(2-3):145-51). Partial agonists of NMDAR, in particular 1-aminocyclopropanecarboxylic acid (ACPC), have been reported to have efficacy in animal models, but have not yet been tested in human studies (Papp M, Moryl E. Antidepressant-like effects of 1-aminocyclopropanecarboxylic acid and D-cycloserine in an animal model of depression. Eur J Pharmacol. 1996; 316(2-3):145-51). Furthermore, effects were only observed at the lowest dose tested, arguing away from high dose treatment in humans. In animal depression models, tolerance over weeks has also been observed, arguing against sustained long term use (Lopes et al., Chronic administration of NMDA glycine partial agonists induces tolerance in the Porsolt swim test. Pharmacol Biochem Behav. 1997; 58(4):1059-64).

D-cycloserine has previously been tested for use in TRD at a dose of 250 mg/day was found to be without significant effect on symptoms of major depression (Heresco-Levy et al., J Affective Disord 93:239-43, 2006). A subsequent study reported significant beneficial effects of D-cycloserine at doses of >500 mg on depressive symptoms when combined with anti-depressant agents (Heresco-Levy et al., Int J Neuropsychopharmacol. 2013; 16:501-506).

As opposed to prior studies in which D-cycloserine was observed to induce psychotomimetic side effects such as paranoia (e.g. Crane G E. Cycloserine as an anti-depressant agent. Am J Psychiatry. 1959; 115:1025-1029; Crane G E. The psychotropic effects of cycloserine: a new use for an antibiotic. Compr Psychiatry. 1961; 2:51-59), no psychotomimetic effects were observed when D-cycloserine was combined with anti-depressants, suggesting an unexpected anti-psychotic effect of anti-depressant agents.

Nevertheless, the prior study did not establish effective dose ranges for D-cycloserine use during clinical treatment of depression.

In general, D-cycloserine has net agonist effects at doses that produce blood concentrations below about 10 microgram/mL, and net antagonist effects at doses that produce concentrations above about 25 microgram/mL. In general, human doses at which D-cycloserine serves as an agonist are in the range of about 10-about 100 mg/day. In general, human doses significantly in excess of about 500 mg/d (e.g. about 700 mg/d; about 10 mg/kg) are needed to achieve sustained blood levels exceeding about 25 microgram/mL.

As described, provided herein is an oral dosage regimen consisting essentially of two active ingredients, wherein a first of said two active ingredients is D-cycloserine formulated to produce sustained plasma D-cycloserine levels of >about 25 microgram/mL and wherein a second of said two active ingredients is a therapeutic agent for the treatment of depression.

In some embodiments, the second therapeutic agent comprises a tetracyclic antidepressant (TeCA), a selective serotonin reuptake inhibitor (SSRI), a serotonin/norephinephrine reuptake inhibitor (SNRI), a norepinephrine reuptake inhibitor (NRI) or a combination thereof.

In some embodiments, the second agent is an atypical antidepressant, a multimodal antidepressant or a low-SERT SNRI.

In some embodiments, the second therapeutic agent is provided at a subtherapeutic dose, if the second therapeutic agent were provided alone.

In some embodiments, this invention provides a method for treating depression in a subject in need thereof, said method comprising providing said subject with an oral dosage regimen as herein described.

In some embodiments, the subject has previously received treatment with an anti-depressant agent.

In some embodiments, the said anti-depressant agent is racemic (S,R) ketamine, S-ketamine or R-ketamine In some embodiments, the anti-depressant agent is an anti-NMDA agent. In some embodiments, the invention provides a method for reducing the incidence or treating suicide or suicide ideation in a subject or population in need thereof, including acute suicidal ideation/the method comprising providing the subject with an oral dosage regimen as herein described.

Surprisingly, the Inventor has found herein that dosages of D-cycloserine produces anti-depressant effects in rodent at plasma levels in excess of about 25 microgram/mL.

It is to be understood that the described compositions are provided by “co-administration” and that the co-administration of either of the two active ingredients to a subject can, in certain embodiments, be combined in a single formulation. In other embodiments, the active ingredients are provided in separate formulations, and which administration can be coincident or staggered.

In some embodiments, when reference is made to an “effective” amount or a “therapeutically effective amount” of D-cycloserine or other therapeutic agents referenced herein, it is meant a nontoxic but sufficient amount of the same to provide the desired effect. In a combination therapy of the present invention, an “effective amount” of one component of the combination is to the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual.

D-cycloserine, or DCS, refers to the chemical D-cycloserine (CA Index Name: 3-Isoxazolidinone, 4-amino-, (4R)-(9CI); CAS Registry No. DCS is an FDA (United States Food and Drug Administration)-approved drug for treatment of tuberculosis, and was developed by Eli Lilly and Company under the trade name Seromycin®.

In some embodiments, the invention provides a regimen further comprising administering a second therapeutic agent for the treatment of depression or for the reduction of the incidence or treatment of suicide or suicide ideation in a subject or population in need thereof.

In some embodiments, the second therapeutic agent comprises a tetracyclic antidepressant (TeCA), selective serotonin reuptake inhibitor (SSRI), a serotonin/norephinephrine reuptake inhibitor (SNRI).

In some embodiments, the second therapeutic agent may comprise any agent as described and/or exemplified herein, for example, the second therapeutic agent may comprise antidepressants, such as monoamine oxidase inhibitors (MAOI), TCAs such as, but not limited to imipramine, amitryptiline, desipramine, clomipramine, TeCAs such as mianserin, mirtazapine, serotonin (SSRI) and serotonin/norephinephrine (SNRI) reuptake inhibitors, such as fluoxetine, fluvoxamine, paroxetine, citalopram, escitalopram, duloxetine, venlafaxine and others, as will be appreciated by the skilled artisan.

In some embodiments, the regimen comprises administering a second therapeutic agent which is an anti-depressant, and said dosage is in accordance with standard prescribing guidelines.

In some embodiments, the regimen comprises a second therapeutic agent which is a psychotropic medication.

In some embodiments, the pharmaceutical compositions can be administered to the patient by any, or a combination, of several routes, for example, whereas D-cycloserine, or alternative NMDAR antagonist may be administered orally, the second therapeutic agent administered, as herein described may be administered by any appropriate route, for example, such second therapeutic agent may be provided as an oral, intravenous, trans-mucosal (e.g. nasal, vaginal, etc.), pulmonary, transdermal, ocular, buccal, sublingual, intraperitoneal, intrathecal, intramuscular, or long term depot preparation.

In some embodiments, this invention contemplates compositions containing both D-cycloserine, or an alternative NMDAR antagonist, and a therapeutic agent for the treatment of depression, suicide or suicide ideation in a subject, where the composition is formulated to produce sustained plasma D-cycloserine levels >25 microgram/mL.

In some embodiments, solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

In some embodiments, liquid compositions for oral administration prepared in water or other aqueous vehicles can include solutions, emulsions, syrups, and elixirs containing, together with the active compound(s), wetting agents, sweeteners, coloring agents, and flavoring agents. Various liquid and powder compositions can be prepared by conventional methods for inhalation into the lungs of the patient to be treated.

In some embodiments, the second therapeutic agent may be formulated as an injectable composition, which may contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

In some embodiments, the second therapeutic agent may be formulated as an intravenous injection, the compounds may be administered by the drip method, whereby a pharmaceutical composition containing the active compound(s) and a physiologically acceptable excipient is infused.

Physiologically acceptable excipients may include, for example, 5% dextrose, to 0.9% saline, Ringer's solution or other suitable excipients. For intramuscular preparations, a sterile composition of a suitable soluble salt form of the compound can be dissolved and administered in a pharmaceutical excipient such as Water-for-Injection, 0.9% saline, or 5% glucose solution, or depot forms of the compounds (e.g., decanoate, palmitate, undecylenic, enanthate) can be dissolved in sesame oil. Alternatively, the pharmaceutical composition can be formulated as a chewing gum, lollipop, or the like.

While the dosage regimen and methods described herein represent an optimum arrived at by administering the D-cycloserine (or alternative NMDAR antagonist) orally, it will be appreciated by the skilled artisan that a lower dosage may be accomplished with the same achieved plasma level by administering the D-cycloserine by a route that does not undergo first pass metabolism. According to this aspect, the dosage can be adjusted to be staggered accordingly, as presented for the oral dosage regimens described herein, with proportionately lower dosages, to accommodate a non-oral administration route, and such alterations are to be considered to be an embodied regimen of this invention.

It will also be appreciated by the skilled artisan that a lower dosage may be accomplished with the same achieved plasma level by administering the D-cycloserine in a formulation that undergoes reduced gastric degradation, for example by applying an enteric coating.

In other embodiments, the formulations as herein described, in particular with regard to oral formulations, are envisaged to comprise slow (sustained) release tablet formulations. Such slow release tablet formulations may, for example, comprise commercially available formulations containing known anti-depressant medications, such as, for example, Effexor® which is already available in extended length (XR) formulations, however the formulation may be modified to further incorporate an NMDAR antagonist.

In other embodiments, the formulations as herein described, in particular with regard to oral formulations, are envisioned to comprise both short acting and extended release formulations. Extended release formulations have the advantage inter alia of minimizing the difference between peak and trough levels of drug, and thereby to increase effectiveness and/or reduce side effects of a medication.

Methods for the formulation of the described regimens herein are well known, and the skilled artisan will appreciate that it is straightforward to prepare the oral dosage regimens as herein described. Applicant, for example, refers to Gibaldi's Drug Delivery Systems in Pharmaceutical Care, Desai A & Lee M (eds), Bethesda, Md.: American Society of Health-System Pharmacists, 2007.

D-cycloserine has a relatively short half-life in human subjects, and therefore is presently used in BID dosing. In some embodiments of the invention BID dosing is envisioned. According to this aspect, and in some embodiments, such consideration will nonetheless ensure that the daily dosage described for the regimens defined herein are not exceeded.

In some embodiments of the invention, D-cycloserine is microencapsulated to increase its circulating half-life. According to this aspect, and in some embodiments, the microencapsulated compound would then be combined either with an anti-depressant medication that is already administered once daily (e.g. sertraline, citalopram, aripiprazole) to insure that cycloserine cannot be taken without accompanying antidepressant (which would increase risk of CNS side effects). Alternatively, the drug could be combined with an anti-depressant compound that is already typically given in divided doses (e.g. venlafaxine, quetiapine) and the two drugs could then be microencapsulated in common to yield a once-daily formulation with similar half-life between the two ingredients. Microencapsulation using standard approaches for (cf. Doshi D H, Oral Drug Delivery Systems, in Gibaldi's Drug Delivery Systems in Pharmaceutical Care, Desai A & Lee M (eds), Bethesda, Md.: American Society of Health-System Pharmacists, 2007. pp. 23-43) such as use of coating materials or matrix-based oral delivery systems. In one approach, for example, drugs are mixed with a gelling agent, such as hydroxypropylmethylcellulose or hydroxylpropylcellulose, which form a hydrophilic matrix (gel) upon contact with water that delays release of the compound. Release properties can be regulated by selection of specific gelling agents, as is known in the art (see, for example, U.S. Pat. No. 5,948,437; European patent EP20040765928, U.S. Pat. No. 7,807,195).

Other compounds that can be used to control release include cellulose, ethylcellulose, gelatin, hypromellose, iron oxide and titanium oxide. In some matrix systems, drug release is controlled mainly by diffusion through matrix pores and not by the erosion of the polymers. Drug delivery can also be controlled by use of reservoir type systems in which release is controlled by osmotic gradient across the coating membrane. Capsules can be manufactured which contain granules with different microencapsulation properties which can be blended to achieve a composition that has a desired release rate. In one embodiment of the invention, D-cycloserine is microencapsulated along with venlafaxine or a pharmaceutically acceptable salt thereof, together with one or more pharmaceutically acceptable excipients. The term gelling agent as used herein means any substance, which forms a gel when in contact with water.

As described, provided herein are methods for treating depression in a subject in need thereof, by administering an effective amount of a composition described herein in the form of an oral or parenteral dosage or a parenteral injection.

In some embodiments, the subject suffers from a depressive disorder, including major depressive disorder or bipolar (e.g., bipolar I or bipolar II) depressive disorder.

In particular embodiments, the described compositions are used in methods for treating Dysthymic disorder. As is known in the art, Dysthymic disorder represents a type of depression in which low-grade symptoms persist for at least 2 years. Other names include dysthymia and pervasive depressive disorder.

In some embodiments, the medicaments in accordance with the described uses of this invention further comprises a second therapeutic agent for the treatment of depression in said subject.

According to this aspect, the method is not limited in terms of the timing of the administration of the second therapeutic agent, such that the methods of this invention contemplate a subject already treated with a second therapeutic agent, or a naive subject concomitantly treated with D-cycloserine and the second therapeutic agent, or in some embodiments, the subject initially treated with D-cycloserine is then administered a second therapeutic agent, and each of these scenarios represents an embodiment of this invention. Such second therapeutic agent will be any such agent as herein described, including a tetracyclic antidepressant (TeCA), selective serotonin reuptake inhibitor (SSRI), a serotonin/norephinephrine reuptake inhibitor (SNRI), an atypical antidepressant, an antipsychotic approved for treatment of depression or a combination thereof.

In some embodiments, in accordance with the methods/uses of this invention, the regimen comprises administering a second therapeutic agent at a dosage which is considered to be suboptimal for treating depression in said subject when treating said subject with said second therapeutic agent alone.

In some embodiments, the method further comprises administering a second therapeutic agent for reducing the incidence or treating suicide or suicide ideation in a subject or population. In some embodiments, the medicaments in accordance with the described uses of this invention further comprises a second therapeutic agent for reducing the incidence or treating suicide or suicide ideation in a subject or population.

According to this aspect, the method is not limited in terms of the timing of the administration of the second therapeutic agent, such that the methods of this invention contemplate a subject or population already treated with a second therapeutic agent, or a naive subject or population concomitantly treated with D-cycloserine and the second therapeutic agent, or in some embodiments, the subject or population is initially treated with D-cycloserine and then administered a second therapeutic agent, and each of these scenarios represents an embodiment of this invention. Such second therapeutic agent will be any such agent as herein described, including a tetracyclic antidepressant (TeCA), selective serotonin reuptake inhibitor (SSRI), a serotonin/norephinephrine reuptake inhibitor (SNRI), an atypical antidepressant or an antipsychotic approved for reducing the incidence or treating suicide or suicide ideation in a subject or population or a combination thereof.

In some embodiments, in accordance with the methods of this invention, the regimen comprises administering a second therapeutic agent at a dosage which is considered to be suboptimal for reducing the incidence or treating suicide or suicide ideation in a subject or population when treating said subject with said second therapeutic agent alone.

As disclosed in Textbook of INTERNAL MEDICINE, Kelley, et al. (eds.), Part X: Neurology, Chapter 469: Major Psychiatric Disorders, (J. Lippincott Co., Philadelphia), pp. 2198-2199 (1992), depression can occur throughout life and is at least twice as common in women as in men. Patients often present without the subjective sense of being depressed but complaining of somatic symptoms of depression, most commonly fatigue, sleep disturbances, or impotence. Patients may describe feeling sad, blue, low, irritable, or anxious, as well as being depressed. Diagnosis of major depression is based either on a distinct change of mood that is prominent, generally persists throughout the day, and occurs each day for at least 2 weeks or on markedly diminished interest or pleasure in most activities over a similar period. The diagnosis requires that at least four of the following symptoms be present nearly every day for a period of 2 weeks: significant weight loss (or weight gain in some younger patients), prominent sleep disturbance, agitation or retardation with slow speech, fatigue, feelings of worthlessness and guilt, slowed thinking, and hopelessness.

Depression can likewise be associated with the symptoms of disease (e.g., systemic lupus erythematosus) or as a side effect of the treatment of disease (e.g., with antihypertensive therapy). One form of depression, postpartum depression, has been commonly found in women during the period following childbirth.

The methods and materials of this invention are therefore suitable for treatment of depression or symptoms of depression associated with other diseases, as herein described.

In some embodiments, according to this aspect, the subject has previously been administered or is concurrently administered a second therapeutic agent for the treatment of depression.

In some embodiments, according to this aspect, the second therapeutic agent comprises any such agent as herein described, for example, a tetracyclic antidepressant (TeCA), selective serotonin reuptake inhibitor (SSRI), a serotonin/norephinephrine reuptake inhibitor (SNRI), a or atypical antidepressant with reduced serotonergic effects, an antipsychotic approved for use in treatment of depression or a combination thereof.

In some embodiments, the second therapeutic agent may comprise any agent as described and/or exemplified herein, for example, monoamine oxidase inhibitors (MAOI) such as, but not limited to isocarboxazid, phenlzine, and selegeline; TCAs such as, but not limited to imipramine, amitryptiline, desipramine, clomipramine; TeCAs such as, but not limited to mianserin, mirtazapine; serotonin (S SRI) and serotonin/norephinephrine (SNRI) reuptake inhibitors, such as, but not limited to sertraline, fluoxetine, citalopram, escitalopram, proxetine, fluvoxamine, trazadone, venlafaxine, desvenlafaxine and duloxetine; an atypical antidepressant such as, but not limited to vilazodone, vortioxetine, milnacipran, and levomilnacipran; an atypical antispychotic such as, but not limited to quetiapine, olanzapine/fluoxetine fixed dose combination, lurasidone, clozapine, ziprasidone, risperidone, asenapine, iloperidone, paliperidone, and cariprazine; and others of the above categories of pharmaceutical agents, as will be appreciated by the skilled artisan.

In some embodiments, according to this aspect, the second therapeutic agent is administered at a dosage, which is considered to be suboptimal for treating depression in said subject when treating said subject with said second therapeutic agent alone.

In some embodiments, according to this aspect, the invention further provides for the use of D-cycloserine in the preparation of a medicament formulated for oral administration at a dosage of >about 500 to <about 1000 mg/day for the treatment of depression in a subject in need thereof, wherein the dose is formulated to produce sustained plasma levels of between about 25-about 35 microgram/mL.

In some embodiments according to the aspect, the invention further provides for the use of D-cycloserine in the preparation of a medicament formulated for oral administration at a dosage of about 10-about 25 mg/kg/day for the treatment of depression in a subject in need thereof, wherein the dose is formulated to produce sustained plasma levels of between about 25-about 35 microgram/mL.

In some embodiments according to the aspect, plasma levels produced by D-cycloserine administration are sustained from about 30 min to about 2 hr following administration.

In some embodiments according to the aspect, plasma levels produced by D-cycloserine administration are sustained from about 30 min to about 12 hr following administration.

A subject undergoing treatment with the methods of the invention can experience significant improvements in depression. Relative to subjects treated with alternative treatments for depression, subjects treated according to the methods of the invention will experience, in some embodiments, greater improvement, or more long-lasting improvement, as measured by any clinically recognized assessment method for depression (e.g., the 21-item Hamilton Depression Rating Scale; the Montgomery-Asberg Depression Rating Scale (MADRS); and the like). It should be noted that not every subject will benefit from the methods of the invention, just as other pharmaceutical agents do not typically benefit every patient.

The following examples describe certain embodiments of the invention and should not be construed as limiting the scope of what is encompassed by the invention in any way.

### EXAMPLES

### Example 1: Dose Dependency of D-cycloserine Antidepression Effects in Rodents

For this study, anti-depressant effects of NMDAR antagonists were assessed using the rodent forced swim tests. NMDAR antagonists were studied alone and in combination with specific 5-HT2A receptor antagonists.

All testing was performed at PsychoGenics Inc, 765 Old Saw Mill River Road, Tarrytown, N.Y. 10591, USA

Male BalbC/J mice (8 weeks old) from Jackson Laboratories (Bar Harbor, Me.) were used. Upon receipt, mice were assigned unique identification numbers (tail marked) and were group housed in OPTImice cages. All animals were acclimated to the colony room for 1 week prior to testing. During the period of acclimation, animals were examined on a regular basis, handled, and weighed to assure adequate health and suitability. Animals were maintained on a 12/12 light/dark cycle. The room temperature was maintained between 20 and 23° C. with a relative humidity maintained between 30% and 70%. Chow and water were provided ad libitum for the duration of the study. All testing was performed during the animal's light cycle phase.

Mice were acclimated to the test room at least 1 hour prior to commencing the test. The forced swimming test consisted of one 6-minute session of forced swimming in individual opaque cylinders (15 cm tall×10 cm wide, 1000 ml beakers) containing fresh tap water at a temperature of 23±2° C. and a depth of 12 cm (approximately 800 ml) for each test animal. The time the animal spent immobile was recorded over the 6 min trial. Every 1 min the cumulative immobility time was recorded from the start of the session and noted on the study data record sheet Immobility was defined as the postural position of floating in the water. The animals were generally observed with the back slightly hunched and the head above water with no movements or with small stabilizing movements of the limbs. After the swim test, each animal was placed in a pre-heated cage with a heating pad and allowed to dry.
Test compounds included:
D-cycloserine (30, 100, 300, 500, and 1000 mg/kg) was dissolved in PTS vehicle (5% PEG200: 5% Tween80: 90% NaCl) and administered PO at a dose volume of 10 mL/kg 30 minutes prior to test.
Sertraline (20 mg/kg) was dissolved in water and administered IP at a dose volume of 10 mL/kg 30 minutes prior to test.

The main variable used to represent immobility in this test was the total time immobile during the 6-min test period. Statistics were performed by analysis of variance (ANOVA) with Dunnett post-hoc testing vs. the control condition or by t-test as appropriate.

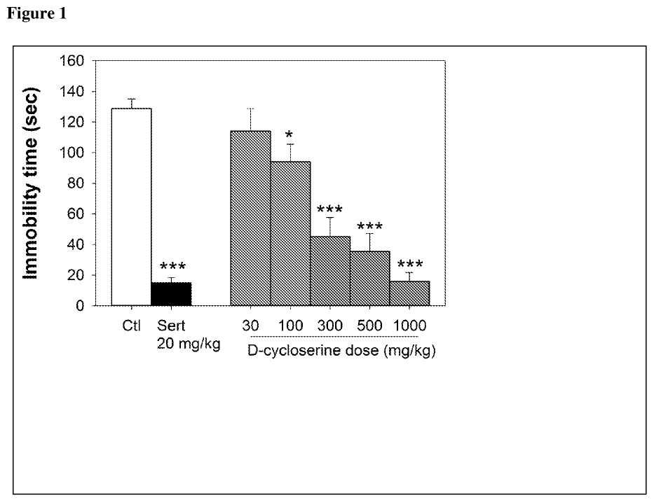

Results are shown in FIG. 1. D-cycloserine (DCS) had no significant effect in the forced swim test assay at a dose of 30 mg/kg. By contrast, D-cycloserine (DCS) had a statistically significant (p<0.001) reduction when administered at doses of 100 mg/kg or above.

### Example 2: Pharmacokinetics of D-cycloserine in Rodents

For this study, the pharmacokinetics of D-cycloserine in rodents were assessed. This study tests the hypothesis that antidepressant effects of DCS presented in the above examples are observed specifically at treatment levels that produce sustained blood DCS levels of >25 microgram/mL.

For this study, male C57BL/6J mice (8 weeks old) from Jackson Laboratories (Bar Harbor, Me.) were used. D-cycloserine (30, 100, 300, 500 and 1000 mg/kg) was dissolved in PTS vehicle (5% PEG200: 5% Tween80: 90% NaCl) and administered IP at a dose volume of 10 mL/kg.

For each treatment group a total of 12 mice were dosed: 4 mice were collected at 30, 60 and 120 min. Mean plasma level was computed over the 30-60 min time period.

Analysis of DCS in plasma was performed utilizing an UPLC/MS/MS system consisted of an Acquity UPLC chromatographic system and a Quattro Premier XE triple quad mass spectrometer, both from Waters. Isolation of DCS was achieved using a 5 minute (total run time) HILIC methodology which provided an LLOQ of 5 ng/mL.

Results of the experiment are shown in Table 1. As shown, peripheral D-cycloserine administration was associated with a dose-dependent increase in plasma D-cycloserine over the 30-60 min time period (p<0.0001). Plasma levels associated with 30 mg/kg DCS treatment were associated with a mean plasma level below 25 microgram/mL. Plasma levels associated with the 100 mg/kg dose or higher were all significantly different from 25 μg/mL (p<0.001). The maximum tolerated dose was 500-1000 mg/kg, suggesting a maximum tolerated blood level of approximately 500 micrograms/mL.

These findings provide a demonstration that blood D-cycloserine levels associated with an antidepression effect in rodents (300 mg/kg) produce plasma levels in excess of 25 microgram/mL.

TABLE 1
Mean plasma levels (microgram/mL) over 30-120 min following
administration of D-cycloserine to rodents

| Dose | | | | p-value vs. |
|---|---|---|---|---|
| (mg/kg) | mean | Std dev | Std err | 25 μg/mL |
| 30 | 24.89 | 4.86 | 2.43 | 0.966 |
| 100 | 74.92 | 7.26 | 3.63 | <0.001 |
| 300 | 251.48 | 28.67 | 14.34 | <0.001 |
| 500 | 375.44 | 25.20 | 12.60 | <0.000 |
| 1000 | 510.73 | 71.56 | 35.78 | <0.001 |

### Example 3: Demonstration of D-Cycloserine Effects in Combination with Anti-Depressants

NRIs, SSRIs, SNRIs, atypical and multimodal antidepressants are all indicated for the treatment of depression. In this example, potential for additive effect was evaluated across a range of anti-depressants. The forced swim test was used as described in Example 1. Compounds tested were as follows:
D-cycloserine (DCS 300 mg/kg) was dissolved in PTS vehicle and administered IP at a dose volume of 10 ml/kg 30 min prior to test.
Bupropion (10 mg/kg) was dissolved in saline and administered IP at a dose volume of 10 mL/kg 30 minutes prior to test.
Desipramine (10 mg/kg) was dissolved in saline and administered IP at a dose volume of 10 mL/kg 30 minutes prior to test.
Imipramine (10 mg/kg) was dissolved in saline and administered IP at a dose volume of 10 mL/kg 30 minutes prior to test.
Sertraline (20 mg/kg) was dissolved in saline and administered IP at a dose volume of 10 mL/kg 30 minutes prior to test.
Venlafaxine (40 mg/kg) was dissolved in saline and administered IP at a dose volume of 10 mL/kg 30 minutes prior to test.
Duloxetine (40 mg/kg) was dissolved in saline and administered IP at a dose volume of 10 mL/kg 30 minutes prior to test.
Fluoxetine (10 mg/kg) was dissolved in saline and administered IP at a dose volume of 10 mL/kg 30 minutes prior to test.
Citalopram (10 mg/kg) was dissolved in saline and administered IP at a dose volume of 10 mL/kg 30 minutes prior to test.
Levomilnacipran (40 mg/kg) was dissolved in sterile water and administered IP at a dose volume of mL/kg 30 minutes prior to test
Milnacipran (40 mg/kg) was dissolved in sterile water and administered IP at a dose volume of mL/kg 30 minutes prior to test
Vilazodone (1 mg/kg) was dissolved in PTS vehicle and administered IP at a dose volume of 10 mL/kg 30 minutes prior to test.
Vortioxetine (10 mg/kg) was dissolved in PTS vehicle and administered IP at a dose volume of 10 mL/kg 30 minutes prior to test.
Mirtazapine (2.5, 5 and 10 mg/kg) were dissolved in PTS vehicle and administered IP at a dose volume of 10 mL/kg 30 minutes prior to test.

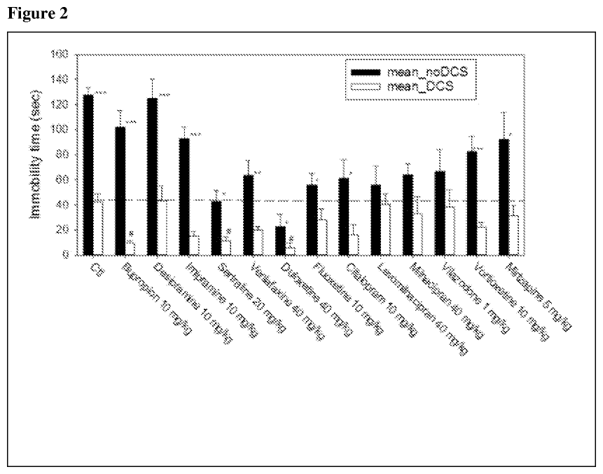

Results are shown in FIG. 2. Effects of DCS 300 mg/kg were highly significant across all agents tested. Particular beneficial effects were observed for bupropion, sertraline, and duloxetine where effects of combined antidepressant and DCS 300 mg/kg were significantly larger than with either agent alone.

### Example 4: Reversal of Rodent Anti-Depression Effects of D-cycloserine by Glycine

Here, the ability of the NMDAR glycine-site agonist glycine to reverse the anti-depression effects induced by D-cycloserine at 300 mg/kg was investigated, which is propose reflect its antagonist action at the NMDAR-associated glycine binding site. NB QX (2,3-dihydroxy-6-nitro-7-sulfamoyl-benzo[f]quinoxaline-2,3-dione) is an antagonist of AMPA receptors and was used as a negative control.

The forced swim test was used as described in Example 1. Compounds were as follows:
D-cycloserine (DSC; 300 mg/kg) was dissolved in PTS vehicle (5% PEG200: 5% Tween80: 90% NaCl) and administered IP at a dose volume of 10 mL/kg 30 minutes prior to test.
Glycine (1.6 g/kg) was dissolved in saline and administered IP at a dose volume of 10 mL/kg 30 minutes prior to DCS or PTS vehicle and 60 minute prior to test.
NBQX (NBQX disodium salt hydrate; Sigma; 10 mg/kg) was dissolved in water and administered IP at a dose volume of 10 mL/kg 30 minutes prior to DCS or PTS vehicle and 60 minutes prior to test.

Results are shown in Table 2. Neither glycine nor NBQX significantly affects immobility time on the forced swim test in the absence of D-cycloserine. However, in the 300 mg/kg condition, glycine significantly reversed D-cycloserine-induced reductions in immobility time. By contrast, NBQX was ineffective.

These data provide a demonstration that anti-depression effects of D-cycloserine formulated to produce blood levels >25 microgram/mL can be reversed by glycine, an agonist at the glycine modulatory site of the NMDAR. These data further provide a demonstration that anti-depression effects of D-cycloserine at 300 mg/kg in rodents reflect its inhibitory effects of D-cycloserine at the glycine modulatory site.

TABLE 2
Effects of glycine (Gly) and NBQX on
DCS-induced anti-depression effects.

| | | | Std. | Std. | P value |
|---|---|---|---|---|---|
| Condition | N | Mean | Deviation | Error | vs. ref cond |
| Control | 50.00 | 128.76 | 44.33 | 6.27 | ref |
| Gly 1.6 g/kg | 10.00 | 129.00 | 53.83 | 17.02 | NS |
| NBQX | 10.00 | 131.60 | 46.25 | 14.63 | NS |
| DCS 300 mg/kg | 40.00 | 44.50 | 51.48 | 8.14 | ref- |
| Glycine + DCS | 10.00 | 76.30 | 49.71 | 15.72 | 0.03 |
| NBQX + DCS | 10.00 | 45.10 | 38.07 | 12.04 | 0.50 |

### Example 5: Combination of D-cycloserine with Lurasidone

Lurasidone is an atypical antipsychotic approved for treatment of bipolar depressive disorder I. Here, the utility of combination treatment between DCS and lurasidone was investigated. Forced swim test measures were as in prior examples. Test compounds were as follows:
D-cycloserine (30 and 300 mg/kg) was dissolved in PTS vehicle (5% PEG200: 5% Tween80: 90% NaCl) and administered IP at a dose volume of 10 mL/kg 30 minutes prior to test
Lurasidone (1 and 3 mg/kg) was dissolved in PTS vehicle (5% PEG200: 5% Tween80: 90% NaCl) and administered IP at a dose volume of 10 mL/kg 30 minutes prior to test.
Lurasidone (1 and 3 mg/kg) and D-cycloserine (30 and 300 mg/kg) was dissolved as a cocktail of both compounds in PTS vehicle and administered IP at a dose volume of 10 mL/kg 30 minutes prior to test.

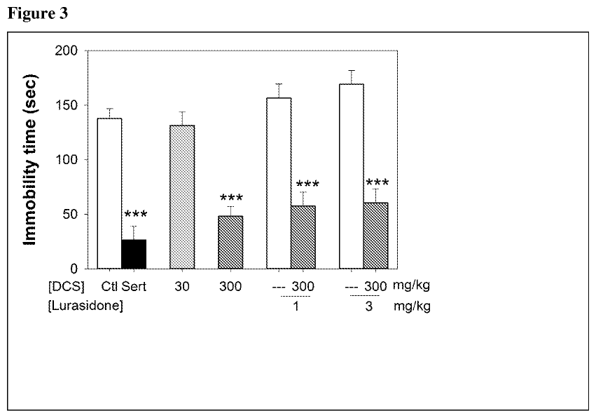

Results are shown in FIG. 3. As in Example 1, DCS had no significant effect at a dose of 30 mg/kg but significantly reduced immobility time at a dose of 300 mg/kg. Lurasidone on its own was without effect in this assay. Combined lurasidone+DCS significantly reduced immobility time vs. control or lurasidone alone, suggesting significant unexpected benefit from this combination.

### Example 6. Combination of DCS with Atypical Antipsychotics Approved for Depression

This example demonstrates anti-depression effects of D-cycloserine in combination with additional atypical antipsychotics approved for treatment of either major or bipolar depression. The rodent forced swim test was used as described in prior examples.
Test compounds were as follows:
D-cycloserine (300 mg/kg) was dissolved in PTS vehicle (5% PEG200: 5% Tween80: 90% NaCl) and administered IP at a dose volume of 10 mL/kg 30 minutes prior to test
Brexpiprazole (0.3 mg/kg) was dissolved in PTS vehicle (5% PEG200: 5% Tween80: 90% NaCl) and administered IP at a dose volume of 10 mL/kg 30 minutes prior to test. In combination studies, Brexpiprazole was given as a cocktail along with DCS.
Aripiprazole (1 mg/kg) was dissolved in PTS vehicle (5% PEG200: 5% Tween80: 90% NaCl) and administered IP at a dose volume of 10 mL/kg 30 minutes prior to test. D-cycloserine (300 mg/kg) was dissolved in PTS vehicle (5% PEG200: 5% Tween80: 90% NaCl) and administered IP at a dose volume of 10 mL/kg 30 minutes prior to test.
Olanzapine (0.6 mg/kg) was dissolved in PTS vehicle and administered IP at a dose volume of 10 mL/kg 30 minutes prior to test.
Quetiapine (10 mg/kg) was dissolved in PTS vehicle and administered IP at a dose volume of 10 mL/kg 30 minutes prior to test.
Risperidone (0.1 mg/kg) was dissolved in PTS vehicle and administered IP at a dose volume of 10 mL/kg 30 minutes prior to test.

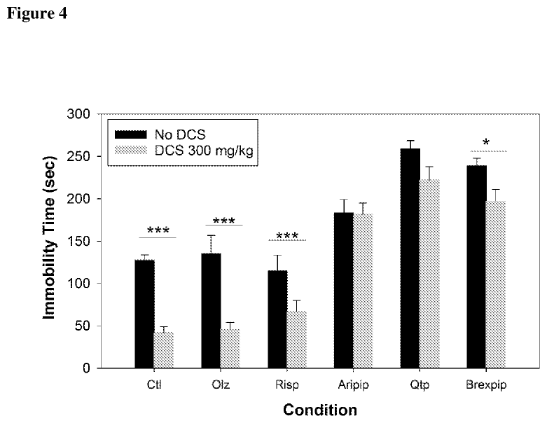

Results are shown in FIG. 4. Significant beneficial effects of DCS 300 mg/kg were observed in the presence of combined olanzapine (Olz) and risperidone (Risp). By contrast, no significant effects were observed in the presence of either aripiprazole (Aripip) or quetiapine (Qtp). Brexpiprazole (Brexpip) on its own increased immobility time. Nevertheless, effects of DCS remained significant in the presence of brexpiprazole. These findings suggest unexpected differential utility of DCS in combination with different antipsychotic agents, and show preferential utility of combinations of DCS with risperidone, olanzapine, and brexpiprazole.

### Example 7: Benefits of Protection of D-cycloserine from Gastric Degradation

Because of its chemical structure, D-cycloserine is susceptible to hydrolysis in aqueous solution under acidic (low pH) conditions, such as may be observed in the stomach, as disclosed by Malspeis and Gold, J Pharmaceutical Sci, 53:113-80, 1964, among others.

Gastric emptying may persist for up to 3 hours, permitting time for hydrolysis of up to 50% of orally administered D-cycloserine.

Enteric coating and prodrug manufacture are known to decrease sensitivity of medications to hydrolysis in the stomach. Examples of prodrugs are disclosed in Fedor et al., Int J Pharmaceutics 22:197-205, 1984, among others. Reduction of hydrolysis by enteric coating or by manufacture of a hydrolysis-resistant prodrug would decrease the oral dose of D-cycloserine needed to achieve the desired plasma levels of >25 microgram/mL.

Effects of different levels of protection from gastric degradation are shown in Table 3, assuming 50% gastric degradation of D-cycloserine if no enteric coating or prodrug modification is present.

TABLE 3
Effects of protection against gastric degradation on
D-cycloserine dose needed to obtain plasma levels in
excess of 25 microgram/mL assuming 50% hydrolysis

| % Protection | Degree of hydrolysis (%) | Dose needed (mg) |
|---|---|---|
| 0 | 50 | 500 mg |
| 50 | 25 | 375 mg |
| 100 | 0 | 250 mg |

This analysis shows the potential benefit of formulation of D-cycloserine for sustained release and protection from gastric degradation.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

All references cited herein, including U.S. patents and published patent applications, international patents and patent applications, and journal references or other publicly available documents, are incorporated herein by reference in their entireties to the same extent as if each reference had been specifically cited for the portion or portions of such reference applicable to the section of this application in which it is cited or to which it is relevant.

I claim:

1. A method for treatment of depression or suicidal ideation comprising:
administering to a subject in need thereof
a first agent comprising D-cycloserine at an antagonist dose; and
a second agent comprising an anti-depressant or atypical antipsychotic, wherein the D-cycloserine is administered at a dose of ≥500 to ≤1000 mg/day, and is formulated to produce an average plasma level in the subject of greater than 25 μg/mL, thereby treating the depression or suicide ideation.

2. The method of claim 1, wherein the depression is major depressive disorder or bipolar disorder.

3. The method of claim 1, wherein administering the first and second agents reduces symptoms of depression, the incidence of suicide, suicide ideation, or a combination thereof.

4. The method of claim 1 wherein the second agent is selected from the group consisting of paroxetine, dapoxetine and indalpine.

5. The method of claim 1, wherein the first and second agents are provided in a single pharmaceutical composition.

6. The method of claim 1, wherein the depression is major depression.

7. The method of claim 1, wherein the second agent is selected from the group consisting of monoamine oxidase inhibitors (MAOI), tricyclic antidepressants (TCAs), tetracyclic antidepressants (TeCAs), norepinephrine-dopamine reuptake inhibitors (NDRIs), selective serotonin reuptake inhibitors (SSRIs), 5-HT2A antagonists, serotonin/norephinephrine (SNRI) reuptake inhibitors and atypical antidepressants.

8. The method of claim 7, wherein the MAOI is selected from the group consisting of isocarboxazid, phenlzine, and selegeline.

9. The method of claim 7, wherein the TCA is selected from the group consisting of imipramine and clomipramine.

10. The method of claim 7, wherein the TeCA is selected from the group consisting of amoxapine, setiptiline, maprotiline, mianserin and mirtazapine.

11. The method of claim 7, wherein the NDRI is selected from the group consisting of bupropion and methylphenidate.

12. The method of claim 7, wherein the SSRI is selected from the group consisting of sertraline, fluoxetine, citalopram, escitalopram, proxetine, fluvoxamine and trazadone.

13. The method of claim 7, wherein the SNRI is selected from the group consisting of venlafaxine, desvenlafaxine and duloxetine.

14. The method of claim 7, wherein the wherein the atypical antidepressant is selected from the group consisting of vilazodone, vortioxetine, milnacipran, and levomilnacipran.

15. The method of claim 1, wherein the atypical antipsychotic is selected from the group consisting of olanzapine, risperidone, and brexpiprazole.

16. The method of claim 1, wherein the subject has depressive or mixed episodes associated with bipolar depression.

17. The method of claim 2, wherein the bipolar disorder is either bipolar type I or bipolar type II depressive disorder.

18. The method of claim 1, wherein the depression is atypical, agitated, or melancholic depression.

19. The method of claim 1 wherein, the depression is dysthymic disorder or is associated with mania.

20. The method of claim 1, wherein first agent further comprises an enteric coating.
